(19) United States
(12) Plant Patent Application Publication
(10) Pub. No.: US 2009/0193548 P1
(43) Pub. Date: Jul. 30, 2009

(54) CHESTNUT PLANT NAMED 'AU ENCORE'

(50) ;

(75) Inventors: W. Alfred Dozier, JR., Opelika, AL (US); J. D. Norton, Opelika, AL (US); Curtis J. Hansen, Opelika, AL (US)

Correspondence Address: HAVERSTOCK & OWENS LLP, 162 N WOLFE ROAD, SUNNYVALE, CA, 94086, US

(73) Assignee: Auburn University, an Alabama Corporation

(21) Appl. No.: 12/012,017

(22) Filed: Jan. 30, 2008

(51) Int. Cl. A01H 5/00

(52) U.S. Cl. PLT/152

(57) ABSTRACT

The disease resistant ‘AU Encore’ seguin offers food availability for wildlife over an extended period. A single plant drops nuts for a 2-3 month period (September 27-November 30). Nut size varies with season and the average weight is between 2 and 3 grams. The plant does not bloom until mid-May, therefore late spring frosts do not damage the flowers. In most seasons, the ‘AU Encore’ seguin cultivar will have 2-3 flushes of vegetative growth. The nut quality is similar to the Chinese chestnut in that it is high in starch and sugar (40-42%) and low in fats. ‘AU Encore’ seguin is an excellent companion cultivar for ‘AU Premier’ seguin since the major nut drop for ‘AU Encore’ seguin occurs after the major nut drop period of ‘AU Premier’ seguin.

### LATIN NAME OF THE GENUS AND SPECIES OF THE PLANT CLAIMED

Castanea P. Mill., Castanea seguinii.

### VARIETY DENOMINATION

‘AU ENCORE’

### BACKGROUND OF THE INVENTION

A Chinese chestnut planting was established at Auburn University, Auburn, Ala., from nuts collected in Hubei Province, P.R. China. Plants were grown in containers under sprinkler irrigation at the main campus and selection were made for dwarfism, precocity, cold hardiness, everbearing, productivity, pest resistance, nut size and quality.

### SUMMARY OF THE INVENTION

The present invention relates to a new and distinct sequin dwarf chestnut cultivar that is precocious, produces a heavy crop annually, begins nut drop about September 27 and continues nut drop through November 30. The small nut size (2.4 g) and continuous nut drop over an extended time makes the ‘AU Encore’ seguin an ideal high energy food for wildlife. The seguin nut size is ideal for consumption by quail and turkey. It produces nuts the year of establishment. The nuts are larger than most seguin chestnuts but not as large as Chinese chestnuts. The majority of the nuts from ‘AU Encore’ seguin drop after the majority of the nuts from ‘AU Premier’ Seguin drop. The ‘AU Premier’ and the ‘AU Encore’ seguins are excellent companion cultivars as they both drop nuts over an extended period but the major nut drop period of the cultivars do not overlap. The plant is not affected by chestnut gall wasp, chestnut blight or leaf spot. ‘AU Premier’ is disclosed in U.S. patent application Ser. No. ______, filed on ______, and entitled “CHESTNUT PLANT NAMED ‘AU Premier”’ [Attorney Docket No. AUB-07700], which is hereby incorporated by reference.

The new cultivar is able to be asexually reproduced by budding or grafting onto a seguin seedling rootstock. The unique characteristics come true to form and are established and transmitted through succeeding asexual propagation.

### BRIEF DESCRIPTION OF THE DRAWINGS

FIG. 1 is a photograph of a tree in bloom of the ‘AU ENCORE’ cultivar.

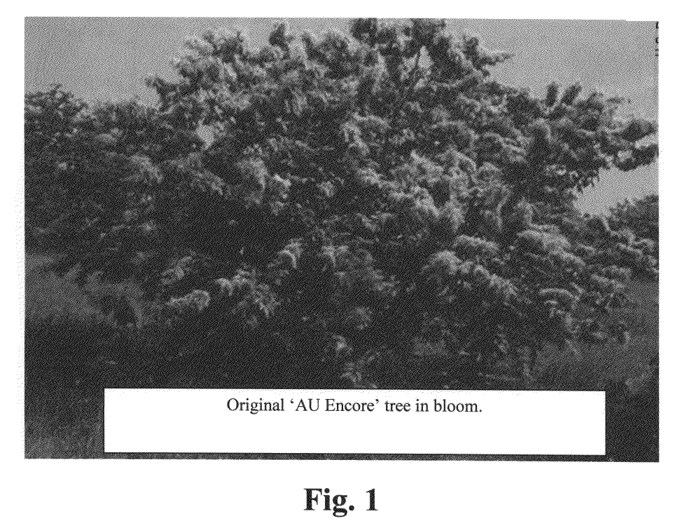

FIG. 2 is a photograph of a bloom on a shoot of the ‘AU ENCORE’ cultivar.

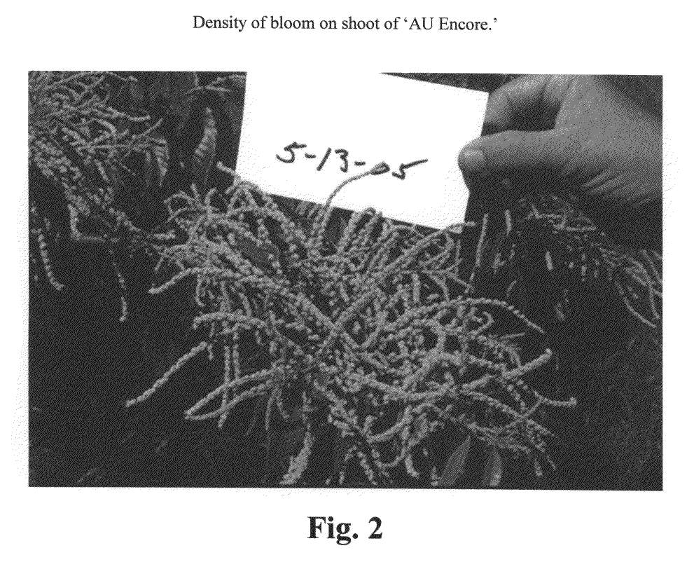

FIG. 3 is a photograph of a tree with fruit of the ‘AU ENCORE’ cultivar.

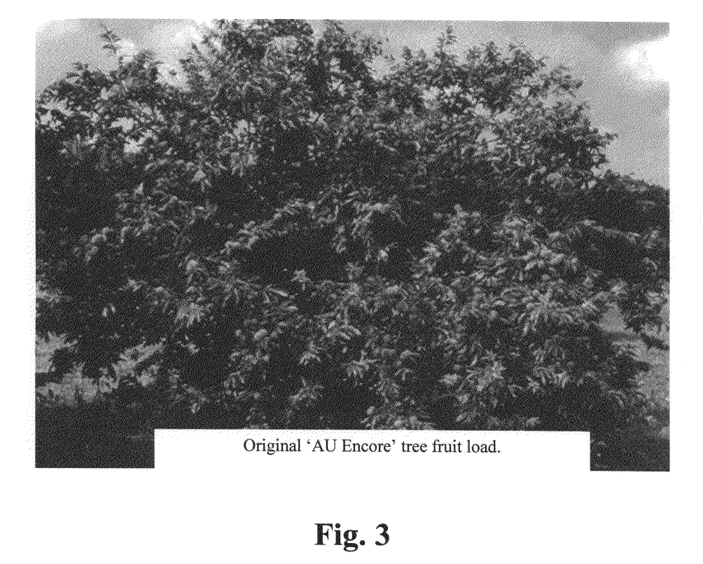

FIG. 4 is a photograph of nuts of the ‘AU ENCORE’ cultivar.

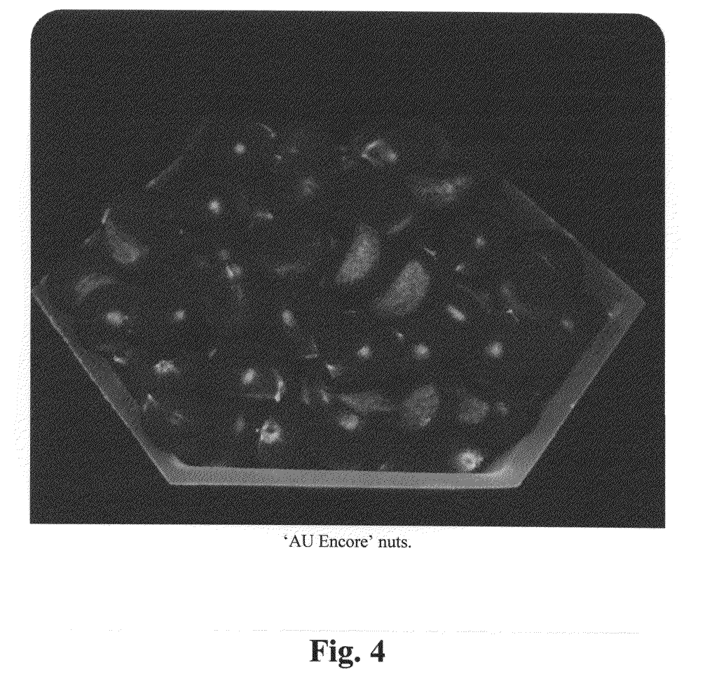

FIG. 5 is a photograph of nuts of the ‘AU ENCORE’ cultivar.

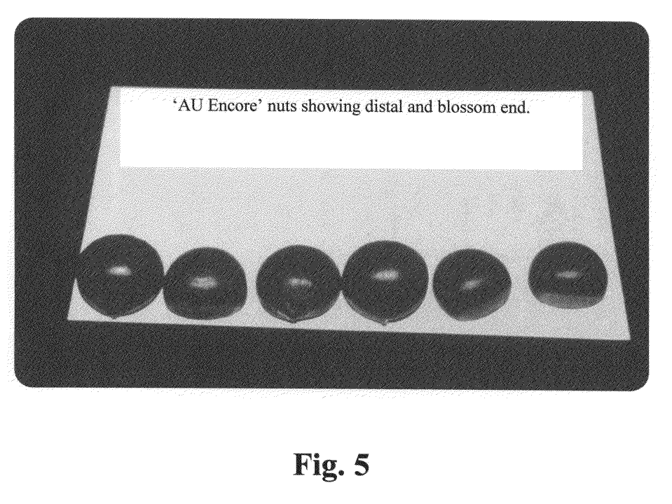

FIG. 6 is a photograph of nuts of the ‘AU PREMIER’ cultivar and the ‘AU ENCORE’ cultivar.

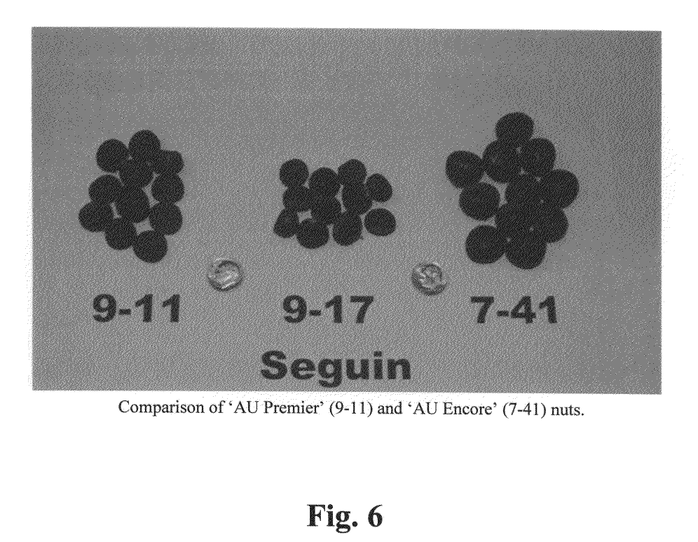

### DETAILED BOTANICAL DESCRIPTION

Seguin chestnut, also spelled “sequin,” is one of two chestnut species, Castanea mollissima and C. seguinii, native to China. It grows as a bush or small tree and is commonly found throughout southeastern and central China. Seguin chestnut is a temperate species and its natural range extends from the Changjiang River region and southeastern China, northward to the southern Hubei province, southward to Guangdong province and westward to Sichuan and Guangxi provinces, a region whose climate is similar to that of the southeastern U.S.A. The plant bears three nuts per bur and the nut size is small (0.5-3 g). It has remained as a noncultivated species in China. The wildly grown nuts and wood are normally harvested by local farmers for food and fuel. The natural range of C. sequinii largely overlaps that of C. mollissima in southeastern and central China. Natural hybridization is able to occur and morphologically distinguishing C. mollissima from seguinni has proven difficult in natural forests. One leaf trait, pubescence on the underside of the leaves, has been studied and used for species identification. Scale-like glandular trichomes are able to be observed on the underside of seguin chestnut leaves with a 10× hand lens, while the underside of Chinese chestnut leaves are pubescent. Despite many efforts to use seguin as a dwarfing rootstock for commercial Chinese chestnut cultivars, it has not been successful due to the complete graft incompatibility between these two species.

Precocity. The plants normally flower at 2-15 months of age after seed germination. It is not unusual for plants to flower as early as three weeks. More than 90% of seedlings produced nuts in the first growing season in Alabama when seeds, introduced from China, were planted. Sprouts resulting from cold damage, pruning or other plant injury bear fruit the first year of development. Plants growing in containers that had the top portion of the plant killed during a snow storm had sprouts develop from the root system and produced a crop of nuts that year. In China, the species is subjected to yearly coppicing in most mountain areas for firewood on which local farmers depend as fuel. The cut off plants develop sprouts from the stump or root system when growth starts in the spring and produces a crop of nuts the same year. The ‘AU Encore’ cultivar produces nuts the first growing season and on multiple vegetative flushes each season and has not exhibited any signs of cold injury.

Everbearing. The continuous flowering throughout the growing season described as ‘everbearing’ is an important characteristic of the seguin chestnut. Twenty percent of plants of two populations collected in Hubei, China, developed bisexual catkins at each new node throughout the growing season. The remaining 80% of the plants were sequential flowering in that the plants produce a set of male and bisexual flowers, after an interval of vegetative growth, a set of flowers develop with each new flush of growth.

‘AU Encore’ is a sequential flowering cultivar. The first burs mature and start dropping nuts during the third to fourth week of September and nuts continue dropping through November. The first bloom occurs in mid-May each season.

The species is resistant to Cryphonectria parasitica, causal agent of chestnut blight. Seguin is generally considered less susceptible to the chestnut gall wasp (Dryocosmus kuriphilus Yasumatsu) than Chinese chestnut because of its growing and flowering habits. No gall wasp damage has been detected on ‘AU Encore’ or any other seguin selections in Auburn tests even though some Chinese chestnut cultivars growing in the same orchard exhibited gall wasp damage.

Some of the original seedlings had a leaf spot problem caused by Colletotrichum gloeosporioides. Infected and defoliated plants were discarded during the recurrent selection program. Leaf spot has not been observed on ‘AU Encore.’

The table below illustrates the specific differences between the ‘AU ENCORE’ cultivar and the ‘REVIVAL’ cultivar.

The botanical details of this new and distinctive variety of
chestnut tree-with color definitions (except those in
common color terms) referenced to Royal Horticultural
Society's Colour Chart (RHS) and color was also determined
using an electronic spectrophotometer to determine hue angle
and chroma (spectrophotometer model CM-2002;
Minolta Camera Co., Japan).
‘AU ENCORE’ CHESTNUT

Tree:
Size (at maturity)-small
Height 4.5 meters, canopy width 6.1 meters, canopy area 42 sq. meters
Vigor-vigorous
Trunk:
Form-trunk upright, tree shape round; branches low and
dense, spreading.
Texture-relatively smooth
Color of bark-Greyed-green, RHS 197A, Chroma C*
14.72, hue angle 85.04
Branches:
Form-strong
Texture-relatively smooth
Lenticels-few, small
Branching habit-low, dense and spreading.
Color-new wood: brown, RHS N200A, Chroma C*
12.15, hue angle 70.82;
mature wood: greyed-green, RHS 197A, Chroma C*9.56, hue angle 90.16
Foliage:
Quantity-abundant
Density-dense
Leaves:
Size-small. Length (cm) 12.3 (10.5-14.5) [20]
width (cm) 5.2 (4.3-6.2) [20]
leaf ratio 2.4 (1.9-3.0) [20]
Shape-elliptic to oblong-elliptic
leaf tip-acuminate
leaf base-cuneate to rounded
Thickness-thin. Leaf venation 1° pinnate: 2° ± parallel, not
prominent abaxially
Texture-weakly coriaceous (thin)
Margin-coarsely serrate, ascending teeth
Petiole-length, short. (cm) 0.6 (0.4-1.0) [20]
Petiole pubescence- glabrous
Color-adaxial surface, glabrous blade, occasionally sparse simple
hairs on main veins. Medium green, moderately shiny, RHS 147A
Chroma C* 13.30, hue angle 118.34
abaxial surface-small scale-like trichomes on blade,
concentrated along midrib, sparse simple hairs on veins
lighter to medium green, RHS 148A, Chroma C* 23.49, hue angle 97.56
Bloom:
Amount of bloom-heavy, at each node on current growth
Color-at anthesis, 161D greyed-yellow group, 157D green-white group,
155C white group
Blooming period-late, full bloom mid-May.
Age at which tree starts flowering-early, first year
Male flower-Catkin length (cm)-7.8 (6.5-9.5) [15]
Male flower-stamen number per catkin-13.2 (10-14) [13]
Female flower-flower number per bur-3.0
Female flower-style number per flower 8.1 (7-9) [8]
Crop:
Bearing-annual, very precocious
Productivity-prolific
Ripening period-late September-late November
Distribution of nuts on tree-well distributed, chain of burs on
all new vegetative growth
Tenacity-burs open while on tree and nuts are easily released and fall.
Hull:
Description-spiny, round bur, average spine length 9.4 mm
Size-(mm) average length 43.7, width 25.5, depth 37.6
Number of nuts-normally 3 per bur
Dehiscence-splits easily and opens wide while still on tree
and after nuts drop the bur is shed
Color-yellow-green at dehiscence, RHS N144C
Nut:
Size-small; average size (mm)-height 17.2, width 17.9; average
weight 2.4 g, average number nuts per pound-189.2
Form-usually 3 in a bur, flattened on 1 or 2 sides, hemispheric;
often bulging sides appearing rounded distally.
Blossom end-little or no tip, distal ⅛ to ¼ end of nut, small
fine white hairs exhibited.
Basal end-flattened
Color-light brown to red brown, RHS 200B, Chroma C*
14.07, hue angle 39.31
Shell-thin
Hardness of shell-relatively hard, yet not rigid
Texture of shell-smooth
Percentage of kernel to nut-high-95% shell out
Kernel:
Size-almost as large as nut size
Form-same as nut shape
Pellicle-thin brown
Flavor-excellent, very sweet
Color-greyed-yellow-RHS 162A, Chroma C* 52.05, hue angle 82.47
Resistance to insects: no insect susceptibilities noted due to bloom period
and development, appears to be resistant to gall wasp damage
Resistance to disease: resistant to chestnut blight
(Cryphonectria parasitica) and leaf spot
(Colletotrichum gloesporioides)
The seguin tree and its nuts herein described may
vary in slight detail due to climatic and soil conditions
under which the variety may be grown; the present
description being of the variety as grown in Camp Hill,
Ala.

The botanical details of this new and distinct variety of
chestnut tree-with color definitions (except those in
common color terms) referenced to Maerz and Paul
Dictionary of Color-are as follows:
‘REVIVAL’

Tree:
Size (at maturity)-large
Vigor-very vigorous
Trunk:
Form-upright with branches spreading in upper
reaches of tree.
Texture-relatively smooth
Color of bark-Silvergray (13-A-1)
Branches:
Form-strong
Texture-relatively smooth
Lenticels-few, small
Branching habit-spreading in upper region of tree
Color-new wood: reddish brown and glossy, mature
wood: silver gray
Foliage:
Quantity-abundant
Density-dense
Leaves:
Size-large. Average length-5-7″ (including petiole).
Average width-2″
Shape-oblong with acute tip and rounded base
Thickness-thick
Texture-smooth
Margin-dentate
Petiole-length medium Thickness: medium.
Color-Top side-glossy dark green (22-L-12).
Under side-lighter green (21-D-7).
Bloom:
Amount of bloom-heavy
Color-cream white (17-B-1)
Blooming period-late. After leaf out in April
Age at which tree starts flowering-early; 2-3 years
years after graft replacement.
Crop:
Bearing-regular (yearly) bearer
Productivity-prolific
Ripening period-short. September 15-October 1.
Distribution of nuts on tree-well distributed
Tenacity-burrs crack while on tree and nuts easily
release, many falling by themselves
Hull:
Description-spiny, round burr
Size-3-4″ in diameter
Number of nuts-2-3 per burr
Dehiscence-splits easily when still on tree. Some
entire burrs split and fall to ground
Color-brown (15-A-8)
Nut:
Size-large. Average size-1⅛″X1 ⅛″X1″ thick.
Average weight-24-32 nuts per pound
Form-broad and ovoid on one side, flat on other side
Blossom end-pointed tip
Basal end-flattened
Color-India Red (7-L-6).
Shell-thin
Hardness of shell-relatively hard, yet not rigid
Texture of shell-smooth
Percentage of kernel to nut-very high (95%)
Kernel:
Size-almost as large as nut size
Form-same as nut shape
Pellicle-thin
Flavor-excellent. Very sweet.
Color-Oyster white (10-B-1)
Resistance to insects: no unusual susceptibilities noted
Resistance to disease: very high inherent resistance to
chestnut bark fungus (Endothia paraslica), no
other susceptibilities to any other disease
The chestnut tree and its nuts herein described may
vary in slight detail due to climatic and soil conditions
under which the variety may be grown; the present
description being of the variety as grown in Alachua,
Fla.

What is claimed is:

1. A new and distinct cultivar of the species Castanea seguinii named ‘AU ENCORE’ as described and illustrated herein.
